(19) United States
(12) Patent Application Publication
(10) Pub. No.: US 2012/0116444 A1
(43) Pub. Date: May 10, 2012

(54) APPARATUSES AND METHODS FOR TREATING WOUNDS

(76) Inventors: Richard C. Zodnik, San Juan Capistrano, CA (US); William W. Chang, Irvine, CA (US); Arthur H. Loussararian, Mission Viejo, CA (US)

(21) Appl. No.: 12/940,855

(22) Filed: Nov. 5, 2010

(51) Int. Cl. A61B 17/135; A61B 17/132

(52) U.S. Cl. 606/202; 606/201

(57) ABSTRACT

A clamp apparatus is particularly configured for controlling bleeding by applying localized pressure to a wound site without strangulating the body member. A restraint supports the body member and inhibits bending thereof. A clear pressure pad having a protuberance is applied to the wound site. The protuberance may include a magnifying lens to magnify the wound site. Slots may be formed in the protuberance and a backing and aligned to receive a needle so that the transparent, magnifying pressure pad may be applied to a patient while undergoing an injective procedure, e.g., angiography. A pressure source provides force to the pressure pad. Strangulation is either prevented by wrapping an inflatable pressure cuff around the restraint or facilitated by wrapping the cuff directly around the body member without a restraint. An inflatable pressure pad is also provided to be applied to a wound site without a restraint.

### BACKGROUND OF THE INVENTION

1. Field of the Invention

The invention relates generally to medical devices and methods, and more particularly to clamps for controlling bleeding.

2. Description of Prior Art and Related Information

A common site used to perform angiography is at the brachial artery in the antecubital fossa adjacent to the elbow since this is a convenient site for gaining access to the great vessels of the circulatory system.

After an angiography is performed via the arm, the points of entry in both the skin and the brachial artery must be compressed sufficiently for a length of time that will allow complete clotting of the exit sites to prevent bleeding out.

Conventionally, this is accomplished by applying manual pressure over the brachial artery in the antecubital fossa until hemostasis is achieved, or by assembling components available in a hospital catheterization laboratory, or “cath lab.” In one common method, a dressing is applied and bandage materials are wrapped around the arm. A blood pressure cuff is applied over the dressing and around the arm, and then inflated until all blood flow is entirely cut off from the application site to the balance of the arm. Thus, pressure is applied not only to the arterial wound site, but also to the periphery of the arm adjacent to the wound site so as to strangulate blood flow entirely, causing cold fingers and extreme pain.

### SUMMARY OF THE INVENTION

In accordance with the present invention, structures and associated methods are disclosed which address these needs and overcome the deficiencies of the prior art.

In one aspect, an apparatus for treating wounds is provided. This clamp apparatus comprises a restraint having a base and a securing mechanism. The base includes an upper surface configured to support a body member capable of bending. The base may comprise a partially circular tube. The securing mechanism is configured to secure the body member to the base to inhibit bending of the body member. The securing mechanism may comprise straps. A pressure pad is configured to apply localized pressure to a wound site. A pressure source is configured to apply force to the protuberance without strangulating the body member. The pressure source may comprise an inflatable pressure cuff wrapped around the pressure pad and the base.

The pressure pad comprises a translucent, preferably transparent, protuberance protruding from a translucent, preferably transparent, plate. A magnifying lens is formed integrally with the protuberance and located at the bottom of the protruding bump. The plate comprises a first proximal slot while the protuberance comprises a second proximal slot aligned with the first slot.

In a further aspect, an apparatus for treating wounds comprises a restraint, a pressure pad, and a pressure source. The restraint includes a base and a securing mechanism. The base includes an upper surface configured to support a body member capable of bending. The securing mechanism is configured to secure the body member to the base to inhibit bending. The securing mechanism may comprise at least one strap.

The pressure pad is configured to apply localized pressure to the body member. The pressure pad preferably comprises a transparent protuberance with a magnifying lens at a bottom tip. The plate comprises a first slot while the protuberance comprises a second slot aligned with the first slot, with both slots aligned to receive a catheter sheath or other injective instrumentation.

The pressure source preferably comprises a circumferential inflatable pressure cuff which can be wrapped around the base so as not to strangulate the body member. The pressure source is configured to apply the protuberance against the body member. The pressure source may comprise a circumferential inflatable pressure cuff.

In a further aspect, an apparatus for treating wounds need not necessarily clamp the body member, but can simply apply localized pressure to a wound site. The apparatus preferably comprises a plate and a translucent, or more preferably transparent, protuberance projecting from the plate. Configured to make contact with a body member or wound site, the protuberance preferably comprises a magnifying lens. The apparatus may further comprise an inflatable pressure cuff configured to apply the protuberance against the body member.

The apparatus can also comprise a restraint having a curved base and a strap. The base may include a curved upper surface configured to support a body member capable of bending. The strap may be configured to secure the body member to the base to inhibit bending and to apply the protuberance to the body member.

An inflatable apparatus for treating wounds is also provided. The apparatus comprises a rigid protuberance having a rounded outer surface configured to face a wound site, and an opposite flat surface. An inflatable pouch is attached to the flat surface of the protuberance and configured to apply force towards the protuberance against the wound site when inflated. An adhesive layer is configured to keep the protuberance pressed against wound site. The apparatus further comprises a pump removably attached to the inflatable pouch. The inflatable pouch may comprise a generally rectangular or circular shape.

A method for controlling bleeding is also provided. The method comprises inhibiting bending of a body member having a wound site, applying localized pressure to the wound site by using an inflatable pressure cuff, and preventing strangulation of the body member. The step of applying localized pressure to the wound site comprises positioning a transparent protuberance over the wound site, wrapping an inflatable pressure cuff over the protuberance, and inflating the pressure cuff to apply protuberance against the wound site.

The method further comprises positioning a transparent pressure pad having a transparent protuberance over a wound site, magnifying the wound site with a magnifying lens included in the protuberance, and positioning slots in the pressure pad over a catheter sheath or other instrumentation in place in the body member.

The step of inhibiting bending of the body part comprises securing the body part with a base and at least one strap. The step of preventing strangulation of the body member comprises wrapping the inflatable pressure cuff over the base.

In a further aspect, a method for controlling bleeding comprises placing a substantially transparent pressure pad with a plate and a protuberance against a wound site with the protuberance facing the wound site, wrapping an inflatable pressure cuff around the pressure pad, and inflating the pressure cuff to cause the pressure pad to apply localized pressure against the wound site.

The method further comprises magnifying the wound site with a magnifying lens included in the protuberance. The method further comprises inhibiting bending of the body part by securing the body part with a base and at least one strap.

### BRIEF DESCRIPTION OF THE DRAWINGS

FIG. 1 is a perspective view of a first preferred embodiment of an apparatus for treating wounds which prevents strangulation of a body member;

FIG. 2 is an exploded, perspective view of a first preferred pressure pad;

FIG. 3 is a perspective view of the first preferred pressure pad;

FIG. 4 is an axial cross-sectional view of the first preferred apparatus as applied to a patient's arm;

FIG. 5 is a transverse cross-sectional view of the first preferred apparatus as applied to the patient's arm;

FIG. 6 is a perspective view of a second preferred pressure pad;

FIG. 7 is an operative, perspective view of the a second preferred embodiment of an apparatus for treating wounds which prevents strangulation of a body member;

FIG. 8 is an operative, axial cross-sectional view of a third preferred embodiment of an apparatus for treating wounds which facilitates strangulation of a body member;

FIG. 9 is an operative, cross-sectional view of a fourth preferred embodiment of an apparatus for treating wounds without a restraint;

FIG. 10 is an operative, perspective view of a fourth preferred embodiment of an apparatus for treating wounds;

FIG. 11 is a perspective view of a fifth preferred embodiment of an apparatus for treating wounds without a restraint; and

FIG. 12 is a diagram illustrating a preferred method of treating a wound site.

The various embodiments of the invention can now be better understood by turning to the following detailed description wherein illustrated embodiments are described. It is to be expressly understood that the illustrated embodiments are set forth as examples and not by way of limitations on the invention as ultimately defined in the claims.

### DETAILED DESCRIPTION OF THE PREFERRED EMBODIMENTS AND BEST MODE OF INVENTION

Various preferred embodiments are described below for treating wounds and controlling bleeding. The preferred embodiments may comprise clamp apparatuses which help control bleeding at a wound site while restraining the corresponding body part. Such clamp apparatuses may be configured to prevent or enable strangulation of the body member. Other preferred embodiments may comprise apparatuses which apply localized pressure to a wound site without any restraints.

As used throughout the specification, the terms “axial” and “longitudinal” refer to a direction along the length of the apparatus or body part to be secured, whereas “transverse” or “lateral” refer to a direction generally perpendicular to the length of the apparatus or body part.

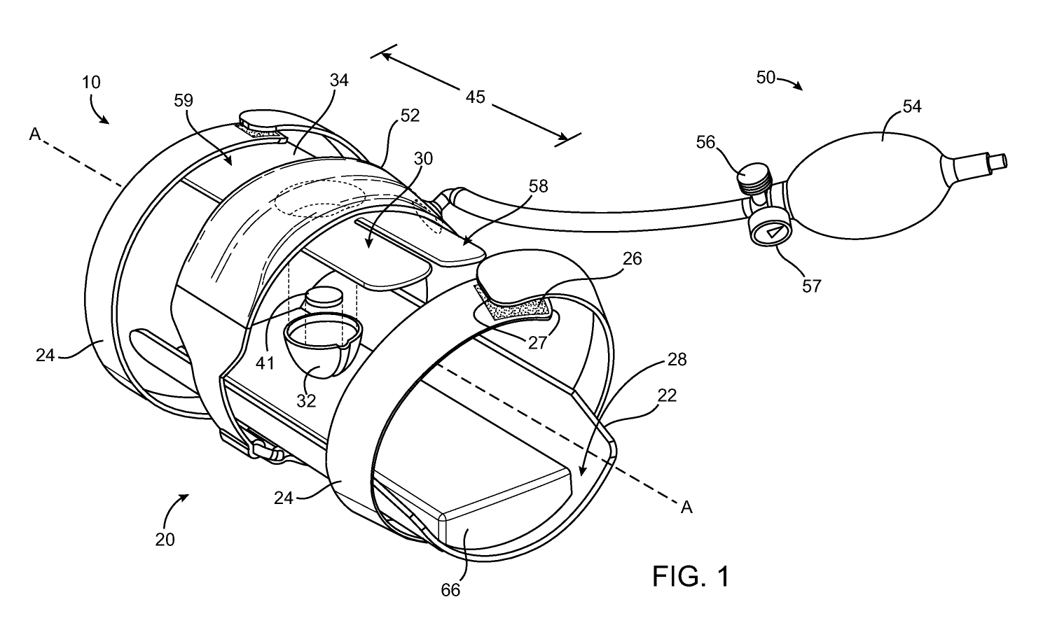

In FIG. 1, a first preferred embodiment of an apparatus for treating wounds comprises a clamp apparatus 10 which prevents strangulation of the corresponding body member. The clamp apparatus 10 is particularly configured for controlling bleeding of wound and/or puncture sites such as at the brachial artery where angiographies are commonly performed. The first preferred apparatus 10 comprises a substantially clear, rigid pressure pad 30, described in further detail below, which helps apply localized pressure to a wound site.

In FIG. 1, the clamp apparatus 10 defines an axis “A” and comprises a restraint 20 configured to secure a body part, particularly a body part capable of bending, such as an elbow. The restraint 20 comprises an elongated base, or support, 22 which is partially curved in the preferred embodiment. The base 22 preferably comprises a profile, meaning that the structure generally maintains the same cross-sectional shape throughout its length. In the preferred embodiment, the base 22 comprises a partially circular profile configured to better support and partially conform around an elbow, for example. In particular, the base 22 preferably comprises a support side 28 that is transversely concave, i.e., curved from side to side.

The restraint 20 comprises a securing mechanism 24 adapted to secure a body part against the base 22 and inhibit bending of the body part. In the preferred embodiment, the securing mechanism 24 preferably comprises a plurality of straps 24 having fastening mechanisms 26 disposed adjacent to the ends 27. In the preferred embodiment, the strap fastening mechanisms 26 may comprise hook-and-loop mechanisms (e.g., Velcro®). While the preferred securing mechanism 24 comprises a plurality of straps, it is to be expressly understood that the securing mechanism may comprise a single strap or wrap. In the first preferred embodiment, the base 22 has a base length 29 in the preferred range of 10 inches to 14 inches.

A first preferred embodiment of the rigid pressure pad 30 is configured to face the wound site when secured by the securing mechanism 24. The pressure pad 30 preferably comprises a clear protuberance, or cup, 32 removably attached to a clear plate, or backing 34. The rounded protuberance, or protrusion, 32 is centrally located with respect to the length 45 of the plate 34 such that a pressure source 50 can be wrapped around the protuberance 32 so to apply pressure.

The pressure source 50 preferably comprises a circumferential inflatable pressure cuff 52, a pressure bulb 54 coupled to the cuff 52, and a control valve 56 for adjusting pressure applied to the wound, and a pressure gauge 57 for measuring the pressure applied. The cuff 52 is preferably adjustable and configured to wrap around protuberance 32 and apply pressure thereto when the bulb 54 is pumped. Outer straps 24 wrap around a proximal plate portion 58 and a distal plate portion 59. While the preferred embodiment comprises a pressure bulb 54, other means of inflating the pressure cuff 52 may also be employed.

It will be appreciated that the preferred clamp apparatus 10 eliminates the strangulation of the arm while allowing the puncture site to safely clot. The interaction between the radius of the restraint 20, the pressure pad 30, and the pressure cuff in our design provides safety and comfort to the patient during the time it takes the puncture to close.

The clamp apparatus 10 may include an optional lower grip mechanism 66 configured to rest on top of the base 22. In the preferred embodiment, the grip mechanism 66 comprises a cushion 66 that provides comfort in supporting a body part, such as an elbow, while providing traction to prevent the body part from slipping away.

In the preferred embodiment, the apparatus 10 is substantially transparent in order to facilitate safety and more precise alignment with a catheter sheath, as well as to observe the wound site for discoloration or bleeding. Accordingly, the base 22, pressure pad 30, and even the securing mechanism 24, including the straps, in the preferred embodiment comprise substantially transparent materials, such as medical grade acrylic.

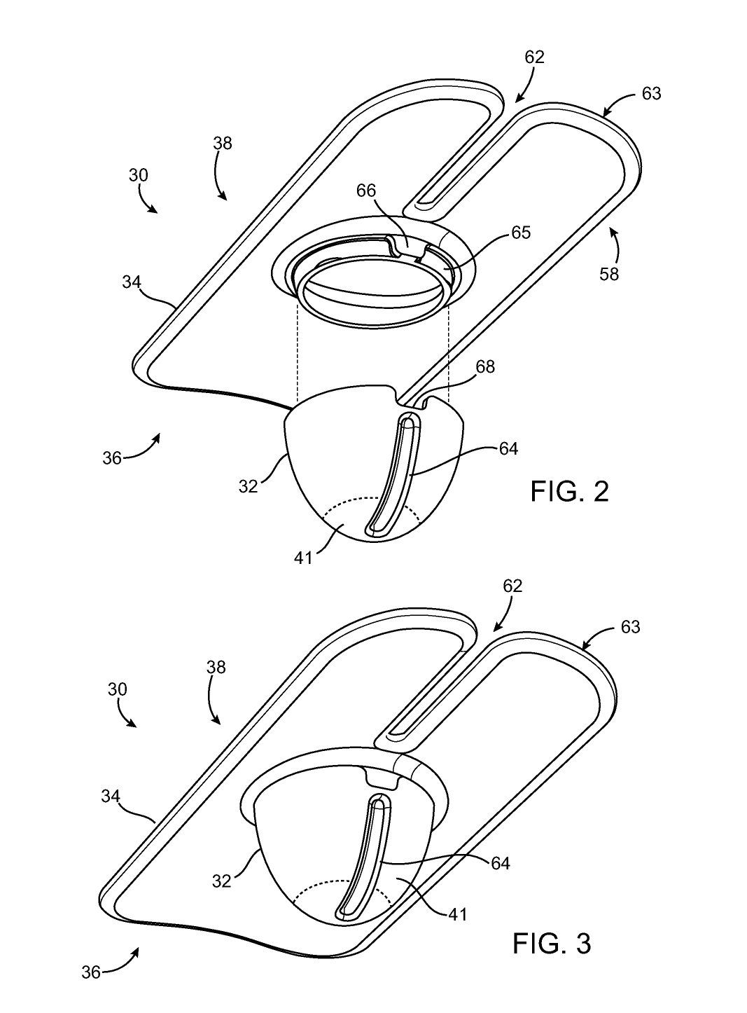

FIGS. 2 and 3 illustrate the first preferred pressure pad 30. In the first preferred embodiment, the pressure pad 30 comprises a rigid, clear protuberance 32 protruding from a clear plate, or backing, 34. Thus, the entire pressure pad 30 is preferably transparent, or at least translucent, to enable one to see through the pad 30. The pressure pad 30 includes a contact side 36 where the protuberance 32 projects from the plate 34 and an opposite viewing side 38. In the preferred embodiment, the protuberance 32 is at least translucent, or more preferably transparent, such that one can view a wound site through the protuberance 32. In the preferred embodiment, the protuberance 32 may be composed of a clear plastic such as medical grade acrylics. The protuberance 32 preferably comprises a magnifying lens 41, shown exploded in FIG. 1, integrally formed therein to enable one to see, from the viewing side 38, a close-up of the target wound site. The magnifying lens 41 is preferably located at the bottom of the protuberance 32.

As an example and not by way of limitation, the protuberance 32 has a depth 43 in the preferred range of 0.5 inch to 2 inches. The plate 34 is preferably elongate and partially curved from side to side to conform over the top of a body part. In the preferred embodiment, the plate 34 is generally rectangular having a length greater 45 than its width 47. As examples and not by way of limitation, the plate length 45 may comprise a preferred range of 6 inches to 10 inches and the width 47 may comprise a preferred range of 2 inches to 3 inches. The plate 34 is preferably curved from side to side, with the contact side 36 being transversely concave so as to accommodate a user's body part, such as the arm.

In the preferred embodiment, a middle proximal slot 62 extends axially along the proximal plate portion 58 from a proximal end 63 of the pressure pad 30 to the protuberance 32. A middle proximal slot 64 is also formed on protuberance 32 and aligned with the plate 62. Accordingly, the protuberance slot 64 and aligned plate slot 62 collectively receive a catheter sheath, needle or other injective instrumentation, and thus enable the clamp apparatus 10 to be applied to a patient even as an angiography or other injective procedure is taking place or being concluded.

Though the protuberance 32 is preferably removable from the plate 34, as better shown in FIG. 2, it is to be expressly understood that the pressure pad 30 may comprise a protuberance that is integral with the plate. In FIG. 2, the plate 34 may comprise a downwardly extending circular rim 65 and a tab 66 configured to mate with a correspondingly shaped groove 68 in the protuberance 32. The protuberance slot 64 is aligned with the groove 68 such that when the protuberance 32 is assembled onto the plate 34, both the protuberance slot 64 and the plate slot 62 are aligned and facing the proximal direction.

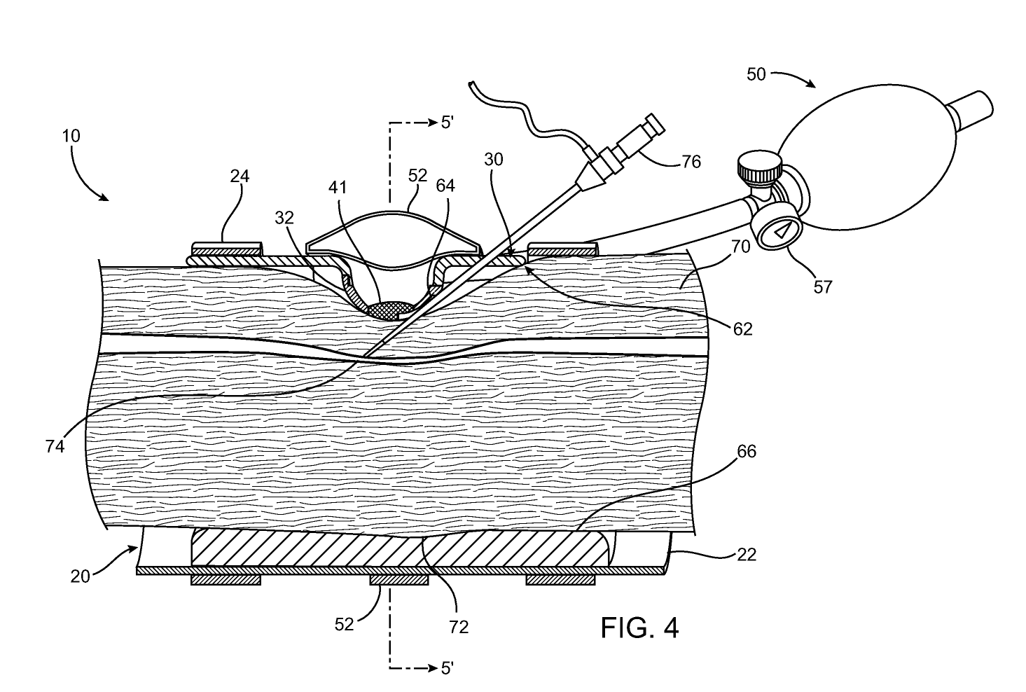

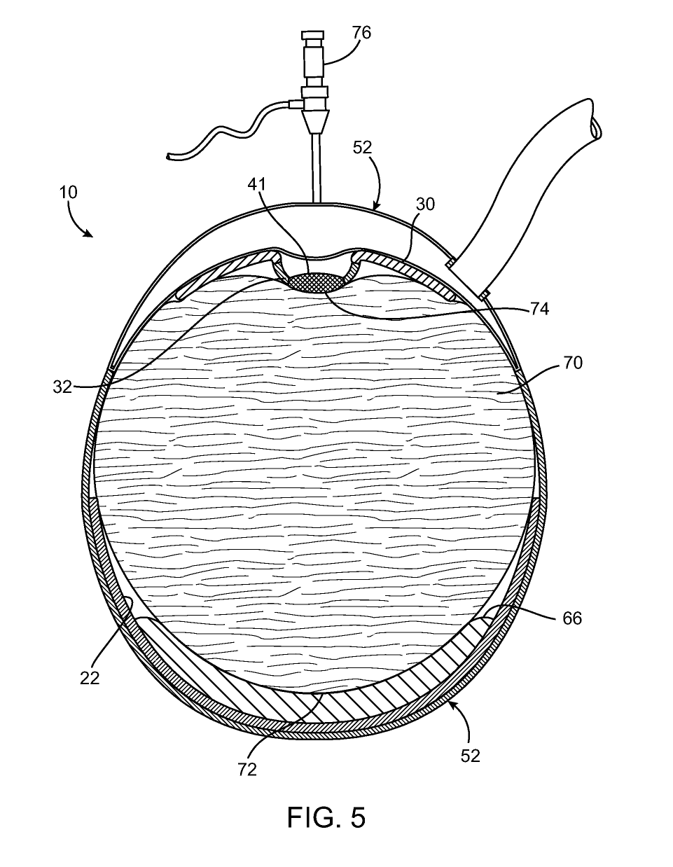

FIGS. 4 and 5 illustrate the first preferred clamp apparatus 10 in use. In particular, the clamp apparatus 10 is applied to a patient's body member 70, such as an arm, even prior to the completion of an injective procedure, such as an angiography. The patient's elbow 72 is placed on the base 22, or on top of the optional cushion 66. The slotted pressure pad 30 is placed over the puncture site 74 with the catheter sheath 76 threaded through the protuberance slot 64 and the plate slot 62, if necessary, as shown in FIG. 4. The straps 24, shown in FIG. 4, are wrapped around the arm 70 and secured. With the protuberance 32 placed over and facing the targeted wound site 74, the inflatable pressure cuff 52 is wrapped around the arm 70, thereby securing the protuberance 32 against or at least adjacent to the wound site.

It will be appreciated that the combination of the magnifying lens 41 and the proximally aligned slots 64, 62 enable the clamp apparatus 10 to be precisely positioned on a target wound site on the arm even while the catheter sheath 76 has yet to be removed. In particular, the plate slot 62 and aligned protuberance slot 64 collectively receive the catheter sheath 76 or any other thin instrument which may be in place in a patient's arm or body member. With the pressure pad 30 being preferably transparent and the magnifying lens 41 providing a close-up view of the target site, the pressure pad 30 may be precisely positioned at the desired location where the protuberance 32 will be most effective in facilitating clotting of the exterior exit wound as well as the interior arterial wound.

The transparency of the pressured pad 30 and the magnification provided by the lens 41 remove any guesswork as to where to apply the localized pressure. Such transparency features in combination with the slots 62, 64 accommodating the catheter sheath 76 not only allow the pressure pad 30 to be placed on the arm while the sheath is still engaged, this unique combination also enables the apparatus 10 to begin applying localized pressure as the sheath is being removed 76.

In FIG. 4, the pressure source 50 may be activated even as the sheath 76 is being removed. With precise positioning of the protuberance 32 enabled by the magnifying lens 41 and sheath accommodating slots 62, 64, localized pressure is applied to the wound site 74 by inflating the pressure cuff 52, thereby causing the protuberance 32 to apply localized pressure against the wound site 74.

It will be appreciated that the base 22, around which the pressure cuff 52 is wrapped, is configured with a diameter or width so as to prevent the cuff 52 from strangulating the arm 70 as shown in FIG. 5. Thus, the restraint 20 serves to support the body member 70, inhibit bending thereof, and prevent strangulation of the body member 70 while pressure is being applied locally to the wound site 74.

Since the first preferred embodiment applies localized pressure against a targeted wound site, such as the brachial artery, the apparatus 10 may comprise a measuring device to provide a precise measurement of the pulse and to secure monitoring of the blood flow in the arm during the procedure. If the apparatus 10 is being used to apply pressure to a brachial artery, then such a measuring device can be applied to the fingers of the patient to measure pulse and blood flow, and thus confirm that blood flow in the brachial artery has been completely occluded.

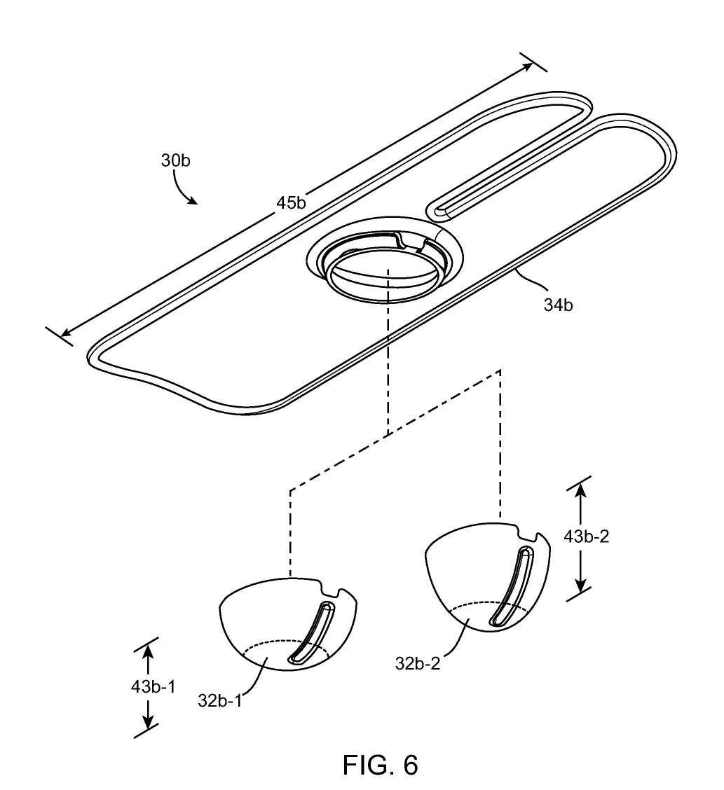

FIG. 6 illustrates a second preferred pressure pad 30b having an elongated plate 34b and comprising a variety of removable protuberances 32b-1, 32b-2 so as to provide adjustability in the depth of the localized pressure desired. In FIG. 6, elements of similar structure and/or function are designated by the same reference numerals followed by the letter b. Here, the pressure pad 30b comprises a plate 34b with an extended length 45b in the preferred range of 10 to 14 inches. By forming the plate 34b with the extended length 45b, the pressure pad 30b can help secure the body part as well as inhibit the bending thereof.

The protuberance is preferably removable from the plate 34b so as to enable a pressure pad 30b to be assembled with different protuberances 32b-1, 32b-2 of varying depths. The first protuberance 32b-1 has a more shallow depth 43b-1 in the range of 0.5 to 1.0 inches whereas the second protuberance 32b-2 has a greater depth 43b-2 in the range of 1.25 to 2.0 inches. It will be appreciated that the pressure pad 30 may be customized by selecting the proper protuberance with the desired depth and assembling the chosen protuberance onto the plate 34b.

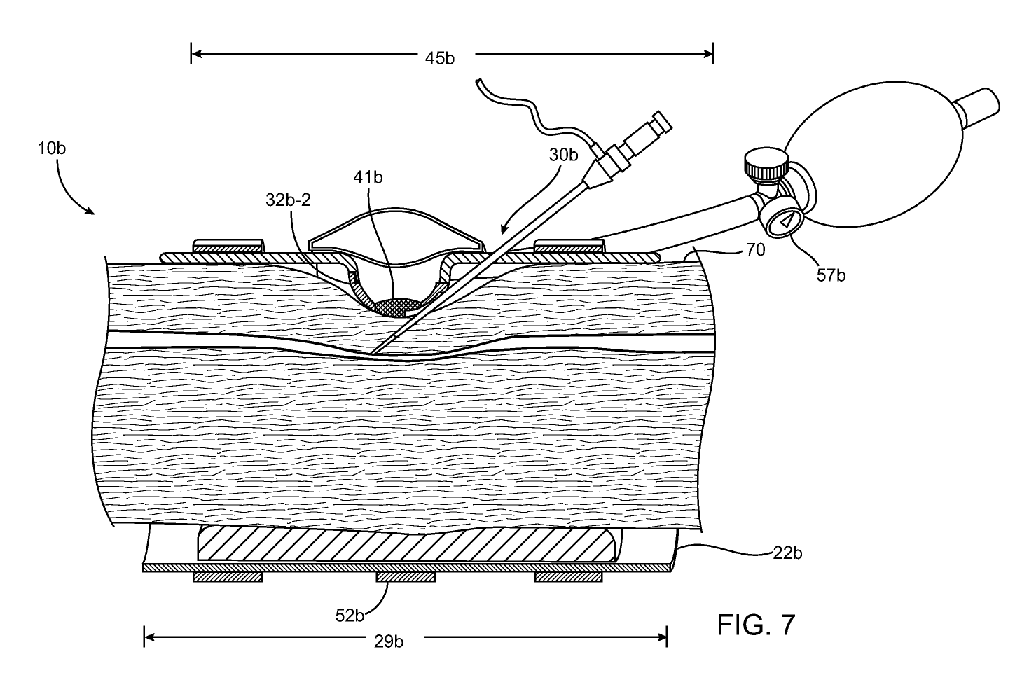

FIG. 7 illustrates a second preferred embodiment of an apparatus for treating wounds which comprises a clamp apparatus 10b utilizing the second preferred pressure pad 30b illustrated in FIG. 6. The second preferred apparatus 10b also prevents strangulation of the body member while applying localized pressure to the wound site. Since the second preferred pressure pad 30b has an extended length and thus facilitates in the securing and straightening of the body member 70, the lower base 22b may have a shortened base length 29b in the preferred range of 4 to 8 inches, such that these upper and lower rigid structures 30b, 22b collectively inhibit bending of the body part. A pressure gauge 57b is provided for measuring pressure applied to the wound.

Though shortened, the base 22b provides the same benefits as the first preferred clamp apparatus 10 in terms of preventing strangulation of the body part 70 by providing a barrier around which the inflatable cuff 52b wraps.

In the illustrated embodiment in FIG. 7, the pressure pad 30b includes a magnifying lens 41b and the deeper protuberance 32b-2 shown in FIG. 6 although it is to be expressly understood that different protuberances of varying depths may be employed.

The first and second preferred embodiments of the apparatus described above apply localized pressure to a wound site while preventing strangulation of the body member. In certain instances, however, it may be desirable to strangulate a body member in addition to applying localized pressure (e.g., to cut off or restrict blood flow to distal portions of a body part).

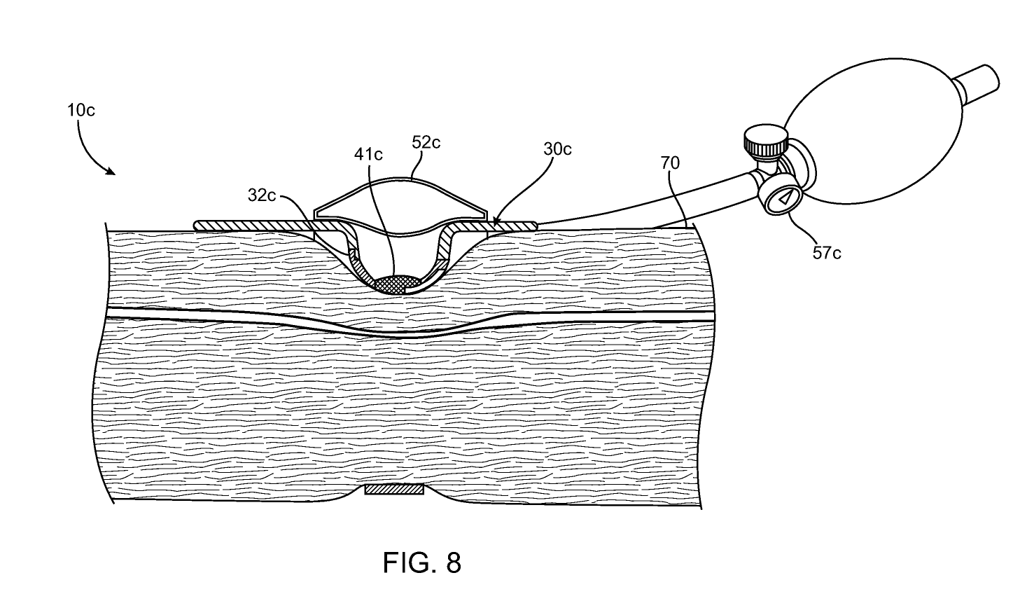

FIG. 8 illustrates a third preferred embodiment of an apparatus for treating wounds 10c which accomplishes both localized pressure and at least partial strangulation of a body member 70. Accordingly, the third preferred clamp apparatus 10c omits the base and associated securing mechanisms shown in the first and second embodiments of the apparatus. The pressure pad 30c in the third preferred apparatus 10c may comprise any of the foregoing pressure pads shown in FIGS. 1-7 and described above, namely, a rigid plate and rigid protuberance, both preferably composed of a substantially clear plastic. A magnifying lens 41c is included at the bottom of the protuberance 32c.

In FIG. 8, an inflatable pressure cuff 52c is wrapped around the patient's body member 70 and over the top of the pressure pad 30c. Since the third preferred apparatus 10c omits a restraint with a base, the pressure cuff 52c applies pressure both to the pressure pad 30c and to the wrapped portion of the body member 70 when the pressure cuff 52c is inflated, thereby strangulating the body member 70 while applying force to the pressure pad 30c. A pressure gauge 57c measures pressure applied.

The preferred embodiments described above help control bleeding and facilitate the clotting of a wound site by applying localized pressure through the combination of a clear pressure pad with a protrusion and a pressure source that is wrapped circumferentially around the corresponding body part. Once clotting has occurred, it may be desirable to remove any restraints and apply lesser pressure to the wound site. Furthermore, it may be impractical or unnecessary in certain instances to wrap a pressure source circumferentially around a body member.

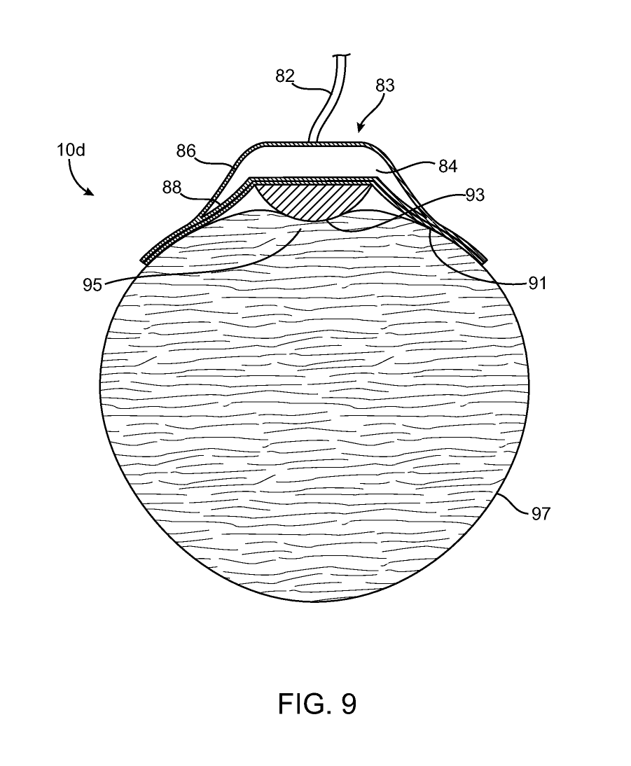

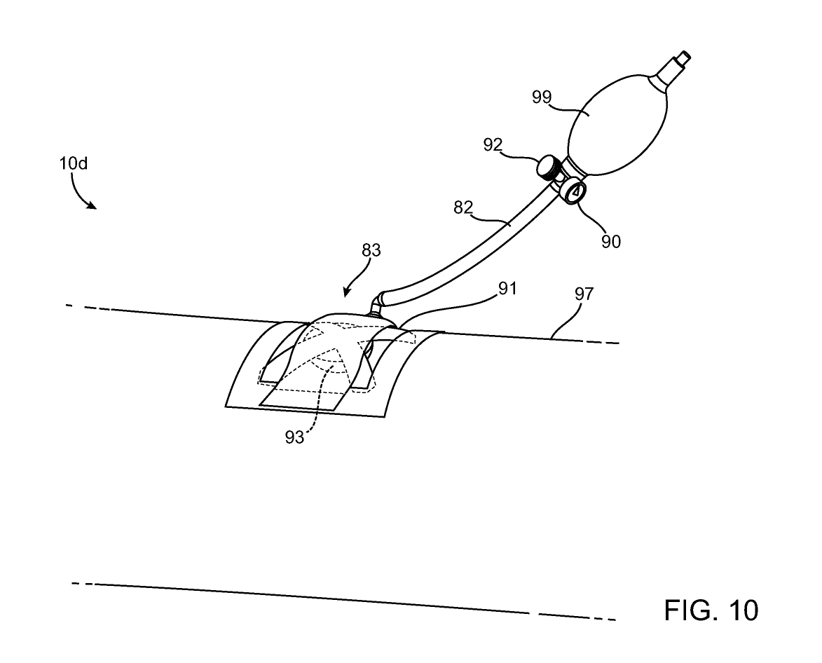

FIGS. 9 and 10 illustrate a fourth preferred embodiment of an apparatus for treating a wound comprising an inflatable pressure pad 10d. In the fourth preferred embodiment, elements of similar structure and/or function are designated by the same reference numerals followed by the letter “d.”

The fourth preferred apparatus 10d is particularly configured to be applied to the arm or body member after one of the above preferred clamp apparatuses, shown in FIGS. 1-8, is removed. Accordingly, the fourth preferred apparatus 10d may be particularly useful to a patient being discharged. Since the fourth preferred apparatus 10d omits restraints, the apparatus 10d may be particularly useful for treating any puncture wound or a procedural puncture site that does not allow a circumferential dressing to be applied.

In FIG. 9, an inflator tube 82 is in fluid communication with an inflatable pouch 83 having a generally rectangular shape and comprising an air chamber 84 defined by an upper layer 86 and lower layer 88. An adhesive layer 91 couples the air chamber 84 on top of a rigid protuberance 93 that is preferably clear and composed of plastic. The protuberance 93 has a rounded outer surface configured to face the wound site, and an opposite rear surface that is substantially flat so as to adhere to, or otherwise, attach to the inflatable pouch 83. The protuberance 93 is then applied over a wound site 95 of a body member 97. The inflator tube 82 may be detachable from the pouch 83.

As the pouch 83 is inflated, such as with a pump 99 shown in FIG. 10, downward force is applied both to the protuberance as well the adhesive layer, thereby causing the adhesive layer to act as a tension source and make contact with the skin. A pressure gauge 90 is provided for measuring pressure applied to the wound. A control valve 92 enables adjustment of the pressure applied.

Since the fourth preferred embodiment 10d does not employ a circumferential pressure cuff or any circumferential restraints, it will be appreciated that this embodiment 10d does not strangulate the body member 97.

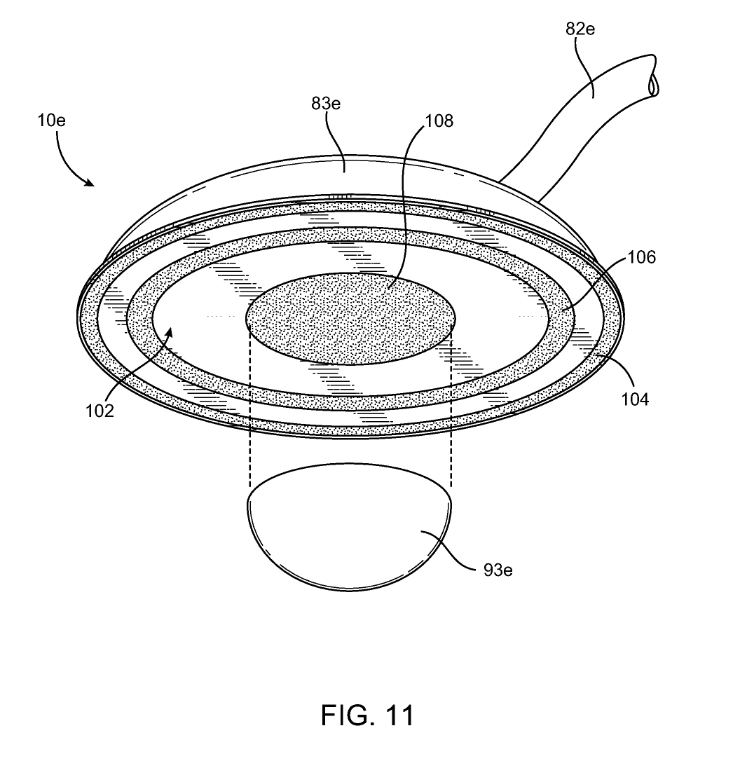

FIG. 11 is a perspective view of a fifth preferred embodiment of an apparatus for treating a wound comprising a semi-spherical inflatable pressure pad 10e. In the fifth preferred embodiment, elements of similar structure and/or function are designated by the same reference numerals followed by the letter “e.”

The fifth preferred apparatus 10e is also particularly configured to be applied to the arm or body member after a preferred clamp apparatus, shown in FIGS. 1-8, is removed. Accordingly, the fifth preferred apparatus 10e may be particularly useful to a patient being discharged. Since the fourth preferred apparatus 10e omits restraints, the apparatus 10e may be particularly useful for treating any puncture wound or a procedural puncture site that does not allow a circumferential dressing to be applied.

In FIG. 11, an inflator tube 82e is in fluid communication with an inflatable semi-spherical pouch 83e comprising an air chamber 84e. Since the inflatable pressure pad has a substantially flat, circular bottom 102, multiple circular adhesive bands 104, 106 may be disposed on the bottom 102. In particular, an outer, peripheral adhesive band 104 is disposed adjacent to the edge of the bottom 102, while an inner concentric adhesive band 106 is disposed within the outer ring 104. The use of multiple, circular adhesive bands 104, 106 help adhere the apparatus 10e onto a user when the pad is inflated. The circular nature of the pad 10e with the circular adhesive bands 104, 106 also help the apparatus 10e stay on the user, and inhibit the accidental or unintentional removal of the apparatus 10e.

A protuberance 93e is centrally located and adhered to the bottom 102 with a circular central adhesive layer 108. Thus, the protuberance 93e is positioned within the adhesive rings 104, 106. The protuberance 93e has a rounded outer surface configured to face the wound site, and an opposite rear surface that is substantially flat so as to adhere to, or otherwise, attach to the inflatable pouch 83. As the pouch 83 is inflated, downward force is applied both to the protuberance 93e as well the adhesive layers 104, 106, thereby causing the adhesive layer 104, 106 to act as a tension source and make contact with the skin.

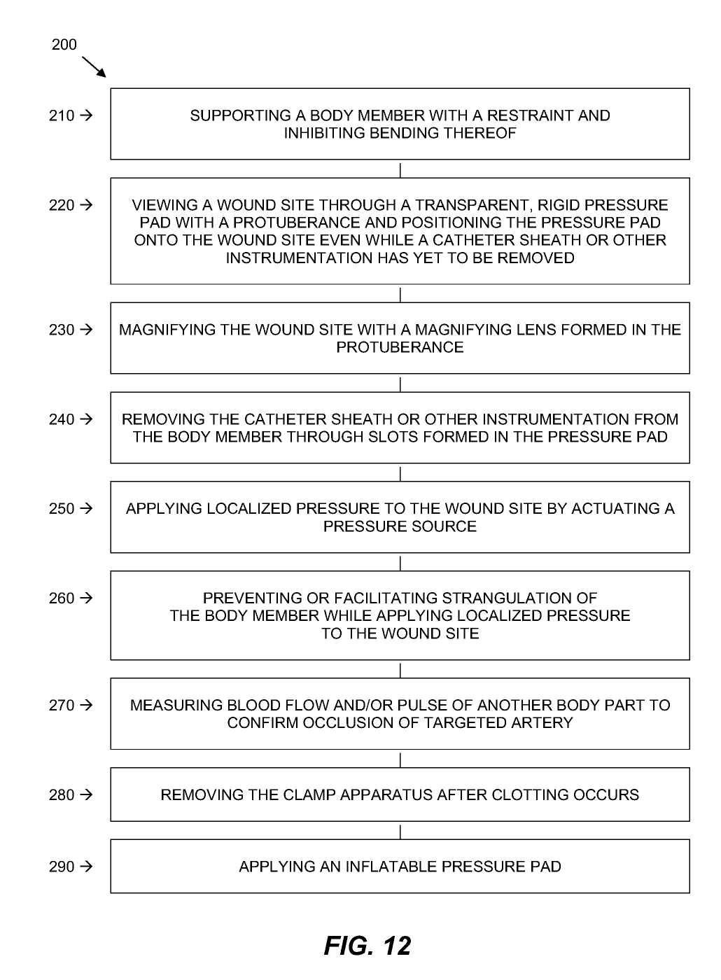

In FIG. 12, a preferred method 200 of treating a wound site is illustrated. In FIG. 12, the method 200 comprises the step 210 of supporting a body member with a restraint. Step 210 may comprise resting the body member on a base and securing the body member to the base with a securing mechanism, such as straps. Step 210 also comprises inhibiting bending of the body member which may be accomplished by strapping the body member to the base as well as applying a pressure pad with an extended length over the top of the wound site.

Step 220 comprises viewing a wound site through a transparent, rigid pressure pad with a protuberance and positioning the pressure pad onto the wound site even while a catheter sheath or other instrumentation has yet to be removed. Step 220 may include applying a clear, rigid pressure pad comprising a protuberance coupled to a plate. Both the protuberance and plate may be slotted in alignment to accommodate a catheter sheath already in place in a patient's arm or body member. Step 220 may comprise placing the pressure pad over the catheter sheath such that proximal, aligned slots formed in the plate and protuberance receive the catheter sheath.

Step 230 comprises magnifying the wound site. This may be accomplished by forming an integral magnifying lens located at the bottom of the protuberance.

Step 240 comprises removing the catheter sheath or other instrumentation from the body member through the slots formed in the pressure pad.

Step 250 comprises applying localized pressure to the wound site by actuating a pressure source which then applies force to the pressure pad. Step 250 may comprise wrapping an inflatable pressure cuff around said pressure pad and the restraint, particularly the base of the restraint. Step 250 then comprises inflating the pressure cuff. Step 250 of applying pressure may occur concurrently with step 240 of removing the catheter sheath. Accordingly, step 240 of removing the sheath need not be completed before beginning step 250 of applying pressure.

Step 260 comprises the step of preventing or facilitating strangulation of the body member while applying localized pressure to the wound site. In the preferred embodiment, step 260 is performed concurrently with step 250 of applying localized pressure. If a circumferential inflatable pressure cuff is employed and strangulation is to be prevented, step 260 comprises wrapping said pressure cuff around the restraint such that the restraint prevents the pressure cuff from strangulating the body member when the pressure cuff is inflated. If strangulation is desired, step 260 may comprise wrapping the pressure cuff around the body member without use of a base or other restraint.

Where the pressure is being applied to an artery, for example, step 270 comprises measuring blood flow or pulse of a specific, distal body member to confirm if the desired conduit has been sufficiently occluded. Step 270 may be accomplished with a device applied to the fingertips, for example, in determining whether the brachial artery has been occluded.

Step 280 comprises removing the preferred clamp apparatus employed in the previous steps, including the restraint, pressure pad and pressure cuff. In the preferred embodiment, this step 280 is performed preferably after clotting at the exit wound site and at any underlying arterial openings is accomplished. Step 280 paves the way for an optional inflatable pressure pad to be applied in step 290.

Step 290 comprises the optional step of applying an inflatable pressure pad without employing a circumferential pressure cuff or circumferential straps. Step 290 may comprise covering a wound site with an inflatable pressure pad. The pressure pad may be adhered directly onto the wound site, or around the site, e.g., with circular adhesive bands which surround the wound site. An inflatable pouch disposed over a rigid protuberance is inflated, applying force on the protuberance against the wound site. The pressure source, which may comprise a bulb or other pump, is preferably removable from the air pouch. Step 290 may also comprise inflating the pressure pad to apply localized pressure to the wound site.

Many alterations and modifications may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The words used in this specification to describe the invention and its various embodiments are to be understood not only in the sense of their commonly defined meanings, but to include by special definition in this specification the generic structure, material or acts of which they represent a single species.

The definitions of the words or elements of the following claims are, therefore, defined in this specification to not only include the combination of elements which are literally set forth. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what incorporates the essential idea of the invention.

What is claimed is:

1. An apparatus for treating wounds, comprising;
a restraint comprising a base and a securing mechanism, the base including an upper surface configured to support a body member capable of bending, the securing mechanism configured to secure the body member to the base to inhibit bending of the body member;
a pressure pad configured to apply localized pressure to a wound site; and
a pressure source configured to apply force to the protuberance without strangulating the body member.

2. The apparatus of claim 1, wherein the pressure pad comprises a protuberance.

3. The apparatus of claim 2, wherein the protuberance is at least translucent.

4. The apparatus of claim 3, wherein the protuberance is transparent.

5. The apparatus of claim 2, wherein the protuberance comprises a magnifying lens.

6. The apparatus of claim 2, wherein the pressure pad comprises a plate, the protuberance protruding from the plate.

7. The apparatus of claim 6, wherein:
the plate comprises a first slot; and
the protuberance comprises a second slot aligned with the first slot.

8. The apparatus of claim 1, wherein the base comprises a partially circular tube.

9. The apparatus of claim 1, wherein the securing mechanism comprises straps.

10. The apparatus of claim 1, wherein the pressure source comprises an inflatable pressure cuff wrapped around the pressure pad and the base.

11. An apparatus for treating wounds, comprising:
a restraint comprising a base and a securing mechanism, the base including an upper surface configured to support a body member capable of bending, the securing mechanism configured to secure the body member to the base to inhibit bending; and
a pressure pad configured to apply localized pressure to the body member;
a pressure source configured to apply the protuberance against the body member, the pressure source being wrapped around the base so as not to strangulate the body member.

12. The apparatus of claim 11, wherein the securing mechanism comprises at least one strap.

13. The apparatus of claim 11, wherein the pressure pad comprises a protuberance.

14. The apparatus of claim 13, wherein:
the plate comprises a first slot; and
the protuberance comprises a second slot aligned with the first slot.

15. The apparatus of claim 11, wherein the protuberance comprises a magnifying lens.

16. The apparatus of claim 11, wherein the protuberance is at least translucent.

17. The apparatus of claim 11, wherein the pressure source comprises an inflatable pressure cuff.

18. The apparatus of claim 11, further comprising a pressure gauge coupled to the pressure source.

19. An apparatus for treating wounds, comprising:
a plate; and
a translucent protuberance projecting from the plate, the protuberance configured to make contact with a body member, the protuberance comprising a magnifying lens.

20. The apparatus of claim 19, further comprising:
an inflatable pressure cuff configured to apply the protuberance against the body member.

21. The apparatus of claim 20, further comprising:
a restraint comprising a curved base and a strap, the base including a curved upper surface configured to support a body member capable of bending, the strap configured to secure the body member to the base to inhibit bending and to apply the protuberance to the body member.

22. An apparatus for treating wounds, comprising:
a rigid protuberance having a rounded outer surface configured to face a wound site and an opposite flat surface;
an inflatable pouch attached to the flat surface of the protuberance and configured to apply force towards the protuberance against the wound site when inflated; and
an adhesive layer configured to keep the protuberance pressed against wound site.

23. The apparatus of claim 22, further comprising a pump removably attached to the inflatable pouch.

24. The apparatus of claim 22, wherein the inflatable pouch comprises a rectangular shape.

25. The apparatus of claim 22, wherein the inflatable pouch comprises a generally circular shape.

26. A method for controlling bleeding, comprising:
inhibiting bending of a body member having a wound site;
applying localized pressure to the wound site by using an inflatable pressure cuff; and
preventing strangulation of the body member.

27. The method of claim 26, wherein applying localized pressure to the wound site comprises:
positioning a transparent protuberance over the wound site;
wrapping an inflatable pressure cuff over the protuberance; and
inflating the pressure cuff to apply protuberance against the wound site.

28. The method of claim 26, further comprising:
positioning a transparent pressure pad having a transparent protuberance over a wound site;
magnifying the wound site with a magnifying lens included in the protuberance; and
positioning slots in the pressure pad over a catheter sheath or other instrumentation in place in the body member.

29. The method of claim 26, wherein inhibiting bending of the body part comprises securing the body part with a base and at least one strap.

30. The method of claim 26, wherein:
inhibiting bending of the body member comprises securing the body member to a base; and
preventing strangulation of the body member comprises wrapping the inflatable pressure cuff over the base.

31. A method for controlling bleeding, comprising:
placing a substantially transparent pressure pad with a plate and a protuberance against a wound site with the protuberance facing the wound site;
wrapping an inflatable pressure cuff around the pressure pad; and
inflating the pressure cuff to cause the pressure pad to apply localized pressure against the wound site.

32. The method of claim 31, further comprising:
magnifying the wound site with a magnifying lens included in the protuberance.

33. The method of claim 31, further comprising:
inhibiting bending of the body part comprises securing the body part with a base and at least one strap.
